SEQUENCE LISTING (4 sequences)

SEQ ID NO: 1 - DNA, 26 bp, Artificial Sequence
  The sequence is synthetized.
caattgtctgatatgggacccacggt

SEQ ID NO: 2 - DNA, 28 bp, Artificial Sequence
  The sequence is synthetized.
atgctgtaccctgcaagagaaatgagac

SEQ ID NO: 3 - DNA, 24 bp, Artificial Sequence
  The sequence is synthetized.
gcagatagcaagcaagctccaacc

SEQ ID NO: 4 - DNA, 24 bp, Artificial Sequence
  The sequence is synthetized.
ggattaacattctcccaccgtcag

(19) United States
(12) Patent Application Publication
(10) Pub. No.: US 2024/0057571 A1
(43) Pub. Date: Feb. 22, 2024

(54) SIMULATION METHOD FOR CHRONIC ATROPHIC GASTRITIS (CAG) LESION AND IDENTIFICATION METHOD FOR MOUSE MODELING

(71) Applicant: CHILDREN'S HOSPITAL OF CHONGQING MEDICAL UNIVERSITY, Chongqing (CN)

(72) Inventors: Yi HUANG, Chongqing (CN); Meihua YANG, Chongqing (CN); Jinmei FENG, Chongqing (CN); Xin ZENG, Chongqing (CN); Xiaohui XU, Chongqing (CN); Jinbao ZANG, Chongqing (CN)

(73) Assignee: CHILDREN'S HOSPITAL OF CHONGQING MEDICAL UNIVERSITY, Chongqing (CN)

(21) Appl. No.: 18/271,677

(22) PCT Filed: May 18, 2022
(86) PCT No.: PCT/CN2022/093473

§ 371 Date: Jul. 11, 2023

(30) Foreign Application Priority Data
Sep. 9, 2021 (CN) 202111059923.0

(51) Int. Cl. A01K 67/027; C12N 15/85

(52) U.S. Cl. CPC A01K 67/0276; A01K 67/0271; C12N 15/8509; A01K 2207/12; A01K 2217/075; A01K 2227/105; A01K 2267/0331; C12N 2015/8536

(57) ABSTRACT

A simulation method for a chronic atrophic gastritis (CAG) lesion includes: (1) taking a metaplasia lesion stage as a simulation object, (2) selecting a simulation form of spasmolytic polypeptide-expressing metaplasia (SPEM), and (3) conditionally deleting gene associated with retinoid-IFN-induced mortality-19 (GRIM-19) from gastric mucosal parietal cells. The present disclosure successfully simulates the SPEM, an initial metaplasia response after a gastric mucosal injury and the initial metaplasia response can progress into intestinal metaplasia (IM) and even gastric cancer (GC) under the continuous stimulation of chronic inflammation. The simulation of this pathological formation provides a basis for research on early prevention and control of intestinal GC and effective suppression of a precancerous lesion of gastric cancer (PLGC), provides a research basis for screening and development of drugs for preventing and treating CAG, and provides an important experimental tool for the implementation of anti-inflammatory and anti-cancer drug tests.

### CROSS REFERENCE TO THE RELATED APPLICATIONS

This application is the national phase entry of International Application No. PCT/CN2022/093473, filed on May 18, 2022, which is based upon and claims priority to Chinese Patent Application No. 202111059923.0, filed on Sep. 9, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBCD140_Sequence_Listing.txt, created on Apr. 21, 2023, and is 1,195 bytes in size.

### TECHNICAL FIELD

The present disclosure belongs to the field of life sciences and specifically relates to a simulation method for a chronic atrophic gastritis (CAG) lesion.

### BACKGROUND

Gastric cancer (GC) is a high-incidence malignant tumor of the digestive tract in China and intestinal GC is the most common subtype of GC, which heavily threatens human health. CAG is a recognized precancerous lesion of intestinal GC and there are many theories about the occurrence of GC. In 1975, Correa proposed a progressive evolution pattern: normal gastric mucosa-chronic superficial gastritis (CSG)-CAG-intestinal metaplasia (IM)-dysplasia (dys)-GC and the accumulation of various genetic and molecular alterations is closely related to this evolution pattern. CAG is a precancerous disease of GC and in particular, CAG accompanied by precancerous lesions of gastric cancer (PLGC) such as IM and dys is highly correlated with the occurrence of GC. Therefore, if CAG and PLGC can be effectively reversed, the incidence rate of GC can be greatly reduced.

In modern experimental research, laboratory animals are widely used to replicate human disease models to investigate the occurrence and development mechanisms and the treatment method of a disease, which is an important basis for experimental medicine. Only when a recognized experimental animal model is selected, an experimental result can have specified credibility, and thus there are high requirements for the similarity of a disease simulated by an experimental animal. How to successfully replicate an ideal experimental animal model of CAG is crucial for research on the prevention, pathogenesis, and reverse therapy of CAG. A variety of CAG models have been constructed:

Chemical modeling method: A chemical reagent is used as a high-risk factor to intervene in a rat, and some chemical reagents include N-methyl-N′-nitro-N-nitrosoguanidine (MNNG), ammonia water, sodium deoxycholate, an antacid, a nonsteroidal anti-inflammatory drug (NSAID), ethanol, and the like. The above reagents can cause repeated chronic injury and inflammatory stimulation, such that an inherent gland of a stomach is destructed to cause atrophy.

Surgical modeling method: Gastrojejunostomy and spring pyloric implantation are commonly used. The surgical modeling method has a short modeling time, but is often affected by a surgical method. The simulated bile reflux cannot be determined, the induced mucosal injuries in rats are inconsistent, and CSG and CAG models often occur simultaneously. Moreover, a high incidence of postoperative infection leads to a high mortality rate for animals.

Biochemical and immunological modeling methods: The onset of chronic atrophic antral gastritis is related to local immune activation. Dai Guanhai et al. successfully replicate a CAG model as follows: a gastric mucosa of the same rat is collected and prepared with normal saline (NS) into a homogenate, the homogenate is mixed with complete freund's adjuvant (CFA) in a ratio of 1:1 to obtain an emulsion, the emulsion is subcutaneously injected into a rat, and stimulation is conducted in combination with sodium deoxycholate and ethanol to obtain the CAG model. Helicobacter pylori (H. pylori) infection is one of the pathogenic factors for CAG. Lee et al. clinically isolate the H. pylori strain SS1, and this strain can effectively colonize in a stomach of an experimental animal for a long time, and successfully cause chronic inflammation, mucosal atrophy, IM, dys, and other pathological changes in the stomach of the experimental animal.

Comprehensive modeling method: Single-factor modeling requires a long modeling time and leads to poor model stability, and multi-factor joint modeling can improve a modeling success rate and shorten a modeling time. Moreover, the pathogeny of CAG is complicated, and thus the comprehensive multi-factor modeling is often used in an experiment to simulate human unhealthy living habits, such that the gastric mucosal injury and repair are conducted repeatedly, which is close to the clinical pathogenesis. Wei Yue et al. establish a rat model with CAG accompanied by Dys as follows: a rat is intragastrically administered with an MNNG solution, allowed to freely drink a 0.1% ammonia water solution, and fed with a 0.03% ranitidine-containing granular feed to obtain the rat model with CAG accompanied by Dys.

Disease-syndrome combination modeling: A CAG disease-syndrome combination model is obtained by constructing a composite traditional Chinese medicine (TCM) syndrome model based on a simple CAG disease model. Chen Xiaoye et al. conduct disease modeling with the combination of active immunization with alternate drinking of a sodium deoxycholate aqueous solution and an aspirin aqueous solution and then conduct spleen deficiency, liver stagnation, and kidney deficiency composite modeling based on CAG molding. Xu Shan et al. replicate a rat CAG disease-syndrome combination model of liver stagnation, spleen deficiency, and dampness-heat, where while a CAG model is replicated, a spleen deficiency CAG group is allowed to undergo bitter cold and diarrhea (intragastric administration of a raw Rheum palmatum decoction every day) and abnormal ingestion (fasting on an odd-numbered day, and adequately feeding on an even-numbered day) from the 7th week to establish a spleen deficiency symptom model.

Although many studies have been conducted on CAG molding, there are still many problems. For example, DMP-777 and L635 intervention methods involve expensive intervention reagents that are difficult to obtain. In tamoxifen modeling, there is a lack of inflammation in gastric mucosa, and it can be spontaneously recovered 3 weeks later, such that the modeling cannot be completed stably. As a class I carcinogen defined by the World Health Organization (WHO), Hp is the most common induction factor for the occurrence of CAG and even GC, but a molecular target of Hp is not clear, and a corresponding model has some limitations in aspects such as molding cycle, molding pathway, and biosafety.

### SUMMARY

In order to solve the problems existing in the prior art, an objective of the present disclosure is to provide a method for efficiently simulating a CAG lesion with a high simulation degree, excellent stability and durability, and specific phenotype.

A simulation method for a CAG lesion is provided, including the following steps:
(1) taking a metaplasia lesion stage as a simulation object;
(2) selecting a simulation form of spasmolytic polypeptide-expressing metaplasia (SPEM); and
(3) conditionally deleting gene associated with retinoid-IFN-induced mortality-19 (GRIM-19) from gastric mucosal parietal cells.

A model obtained by the simulation method of the present disclosure undergoes a spontaneous SPEM lesion in a gastric mucosa at an age of 8 months and can stably express a specific phenotype without repetition.

Further, the simulation method includes: intercrossing a GRIM-19 fox gene-edited mouse GRIM-19^{flox/flox} and an ATP4b-Cre gene-edited mouse for the first time to obtain a gastric mucosa-specific parietal cell GRIM-19-knockout mouse strain (GRIM-19^{-/-}/ATP4b-Cre, SPF grade).

Specifically, a construction method of the gastric mucosa-specific parietal cell GRIM-19-knockout mouse strain includes the following steps:
(1) determining a target gene: determining 5 exons (from an ATG start codon in exon 2 to a TAG stop codon in exon 5) of mouse chromosome 8 at which mouse GRIM-19 (GenBank accession number: NM_023312.2: Ensembl: ENSMUSG00000036199) is located, and selecting exon 3 as a conditional knockout region, where deletion of exon 3 will lead to the loss of a function of the GRIM-19 gene;
(2) designing and constructing a targeting vector plasmid: providing a bacterial artificial chromosome (BAC) clone RP23-74A9 or RP23-114L20 from a C57BL/6J library as a template, preparing a homology arm and a conditional knockout (CKO) region through polymerase chain reaction (PCR); and in a targeting vector, providing a flank of a neomycin (NEO) cassette as a Frt site and a flank of the CKO region as a LoxP site, using diphtheria toxin A (DTA) for negative selection;
(3) subjecting an embryonic stem (ES) cell to electroporation and positive clone screening, microinjecting a resulting ES cell into a mouse to prepare a chimera mouse, intercrossing the chimera mouse and a flp mouse, and deleting a neomycin (NEO) resistance gene to obtain a GRIM-19^{flox/-}F1 mouse; and subjecting the mouse to propagation to obtain an offspring mouse, identifying, and raising the offspring mouse together with a wild-type (WT) mouse C57B/L6 in a same cage to allow propagation to obtain a GRIM-19^{flox/flox} homozygous mouse; and
(4) intercrossing the GRIM-19^{flox/flox} mouse and an ATP4b-cre mouse in a cage, identifying to obtain a GRIM-19^{flox/flox}/ATP4b-cre mouse, and establishing a mouse strain GRIM-19^{-/-}/ATP4b-cre.

In this way, the target gene is accurately deleted.

The present disclosure also provides an identification method for a construction result of the simulation method, with high stability and prominent accuracy.

An identification method for simulated modeling of a CAG lesion is provided, including: identifying a modeled animal through PCR with the following primer pairs: F: 5′-CAATTGTCTGATATGGGACCCACGGT-3′; and R: 5′-ATGCTGTACCCTGCAAGAGAAATGAGAC-3′.

The present disclosure also provides a new use of GRIM-19.

A GRIM-19 gene or protein is used as a drug target or a diagnostic target in preparation of a drug or reagent for treating, preventing, and diagnosing a CAG lesion or an SPEM lesion. The drug or reagent refers to a drug or reagent that can regulate or detect an expression level of GRIM-19. The drug refers to a drug that can enhance an activity of GRIM-19. The present disclosure also provides a use of the GRIM-19 gene in construction of a spontaneous SPEM animal model.

### Beneficial Effects

1. In the present disclosure, since CAG has the main clinical pathological features of “inflammation→atrophy→metaplasia→dys”, metaplasia is adopted as a simulation object for CAG progression. SPEM with the expression of trefoil factor 2 (TFF2) in a deep gastric antral-type gland as a main feature is adopted as a specific simulation form to act on a gastric mucosal cell line. Specifically, a parietal cell defect is used to simulate oxyntic gland atrophy, and specific deletion of GRIM-19 gene in parietal cells is used to achieve this process. The present disclosure successfully simulates an initial metaplasia response after a gastric mucosal injury, and the initial metaplasia response can progress into IM and even GC under the continuous stimulation of chronic inflammation. The simulation of this pathological formation provides a basis for research on early prevention and control of intestinal GC and effective suppression of PLGC, provides a research basis for screening and development of drugs for preventing and treating CAG, and provides an important experimental tool for the implementation of anti-inflammatory and anti-cancer drug tests.

2. The present disclosure creatively discovers that the GRIM-19 protein is involved in the regulation of a pathological process of precancerous inflammation of GC and reveals for the first time that the deletion of the GRIM-19 gene in gastric mucosal parietal cells of an animal will cause mucosal inflammation and SPEM. A targeted defect of gastric mucosal parietal cells is realized through specific deletion of the GRIM-19 gene, thereby achieving the high-degree simulation of a non-infectious spontaneous SPEM lesion. The GRIM-19 protein, also known as NDUFA13 protein, is an essential component of a mitochondrial respiratory chain (MRC) complex I, and its gene is mainly localized on human chromosome 19 19p13.2. The gene encodes a protein molecule with 144 amino acids, which has a molecular weight of about 16 kDa and is mainly localized at an inner mitochondrial membrane. There is also a small amount of the protein molecule in the cytoplasm and nucleus.

### BRIEF DESCRIPTION OF THE DRAWINGS

FIGS. 1A-1B show the construction of a GRIM-19 gene-knockout vector, where FIG. 1A shows the construction of a recombinant targeting vector; and FIG. 1B shows a design strategy for recombinant identification primers.

FIGS. 2A-2B show the gene identification of a parietal cell-specific GRIM-19 gene-knockout mouse, where FIG. 2A is a schematic diagram of hybridization of GRIM-19^{flox/flox} with ATP4b-cre; and FIG. 2B shows the identification of a genotype of a GRIM-19^{flox/flox}/ATP4b-cre mouse.

FIGS. 3A-3B show the analysis results of expression of the GRIM-19 protein in a gastric mucosal tissue of a parietal cell-specific gene-knockout mouse, where FIG. 3A shows an expression level of the GRIM-19 protein in a gastric mucosal tissue detected by immunofluorescence assay (IFA) (scale: 50 μm); FIG. 3B shows the quantitative analysis of mean fluorescence intensity (MFI) (***<0.001); and FIG. 3C shows an expression level of the GRIM-19 protein in a gastric mucosal tissue detected by Western blot (WB) (with β-actin as an internal reference).

FIG. 4 shows hematoxylin and eosin (H&E) staining results of a gastric mucosal tissue of a parietal cell-specific GRIM-19 gene-knockout mouse (100× scale: 200 μm; and 400× scale: 50 μm).

FIG. 5 shows the analysis results of IM markers in a gastric mucosal tissue of a parietal cell-specific GRIM-19 gene-knockout mouse, where expression levels of CDX2, Villin-1, and GKLF4 proteins in the gastric mucosal tissue are detected by WB (with β-actin as an internal reference).

FIG. 6 shows the analysis results of SPEM markers in a gastric mucosal tissue of a parietal cell-specific gene-knockout mouse, where expression levels of TFF2, Mist1, Clusterin-1, HE4, and MUC6 proteins in the gastric mucosal tissue are detected by WB (with β-actin as an internal reference).

FIGS. 7A-7B show GIF and GSII double immunofluorescence assay (DIFA) results of a gastric mucosal tissue of an 8-month-old parietal cell-specific gene-knockout mouse, where FIG. 7A shows the GIF and GSII DIFA results (scale: 50 μm); and FIG. 7B shows the quantitative analysis of MFI (***<0.001).

FIGS. 8A-8B show GIF and GSII DIFA results of a gastric mucosal tissue of a 4-month-old parietal cell-specific gene-knockout mouse, where FIG. 8A shows the GIF and GSII DIFA results (scale: 50 μm); and FIG. 8B shows the quantitative analysis of MFI (***<0.001).

FIGS. 9A-9D shows the analysis results of SPEM markers in a gastric mucosal tissue of a mouse intervened with MCC950, where FIG. 9A shows expression levels of TFF2, Mist1, Clusterin-1, HE4, and MUC6 proteins in the gastric mucosal tissue detected by WB (with β-actin as an internal reference); FIG. 9B shows GIF, GSII, and Mist1 triple immunofluorescence assay (TIFA) results (scale: 50 μm); FIG. 9C shows the GIF+/GSII+double-positive cell counts; and FIG. 9D shows the quantitative analysis of MFI (***<0.001).

### DETAILED DESCRIPTION OF THE EMBODIMENTS

The present disclosure is described in detail below with reference to examples. It should be noted that the examples should not be construed as limiting the protection scope of the present disclosure, and some non-essential improvements and adjustments made to the present disclosure by those skilled in the art based on the content of the present disclosure should still fall within the protection scope of the present disclosure.

### Example 1 Construction of a Gastric Mucosal Parietal Cell-Specific GRIM-19 Gene-Knockout Mouse Model

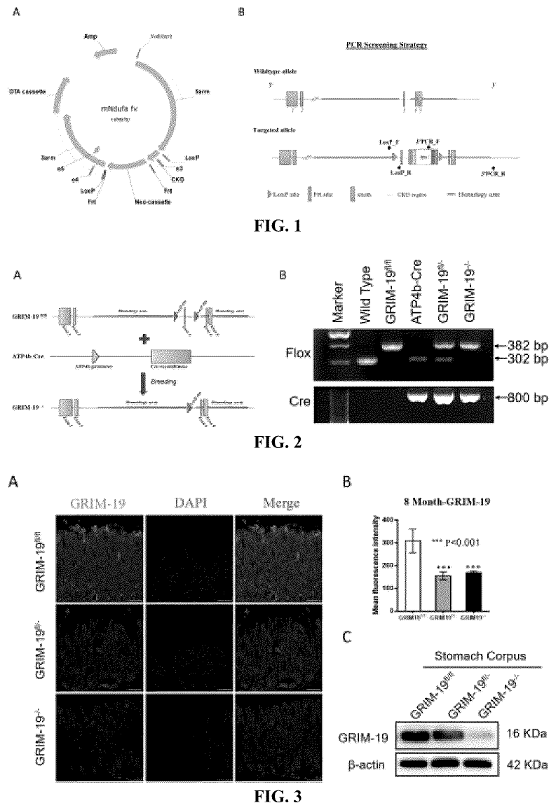

1. A GRIM-19 gene-knockout vector was constructed. As shown in FIG. 1A, a conditional gene targeting method was used to construct the GRIM-19 gene-knockout vector, where since a GRIM-19 target vector included a LoxP site, a LoxP-neo fragment was used to replace exon 3 of a GRIM-19 gene, that is, the exon 3 was deleted.

2. A GRIM-19 gene-knockout mouse was constructed. After the GRIM-19 gene-knockout vector was constructed, the GRIM-19 gene-knockout vector was transfected into a mouse ES cell by electrotransfection, then the ES cell carrying the GRIM-19 gene-knockout vector was injected into an embryonic sac with a C57/B6 background, and then the embryonic sac was implanted into an uterus of a pseudopregnant female mouse to produce a chimera mouse; and the chimera mouse and a flp mouse were intercrossed to delete a NEO resistance gene to produce an F1 generation Loxp-GRIM-19-carried mouse, and the F1 generation Loxp-GRIM-19-carried mouse was raised together with C57B/L6 in a cage to allow propagation for subsequent experiments. A design strategy for recombinant identification primers was shown in FIG. 1B.

3. The GRIM-19^{flox/flox} mouse was raised together with an ATP4b-cre mouse in a same cage to obtain a GRIM-19^{-/-}/ATP4b-cre mouse.

4. Identification of a genotype of the GRIM-19^{-/-}/ATP4b-cre mouse: (1) An experimenter wore sterile gloves, a sterile mask, a sterile hat, and a sterile clothing, and then entered an SPF-level animal laboratory. An ear tag was attached by a sterilized instrument to the mouse, 3 mm to 5 mm of a mouse tail was collected into a 1.5 mL EP tube correspondingly numbered, 200 μL of a lysis buffer (10 μm Tris-HCl pH 8.0, 10 μm EDTA, 15 mM NaCl, and 0.5% SDS) and 4 μL of proteinase k were added, and a resulting mixture was subjected to a digestion reaction overnight at 55° C. A resulting reaction system was centrifuged at 14,000 g for 10 minutes, 100 μL of a resulting supernatant was collected and thoroughly mixed with an equal volume of isopropyl alcohol (IPA), and a resulting mixture was vortexed for 10 s to 20 s and then centrifuged at 14,000 g for 15 minutes. A resulting supernatant was discarded, 75% alcohol was added to clean a resulting precipitate, and a resulting mixture was vortexed for 10 s to 20 s and then centrifuged at 14,000 g for 10 minutes. A resulting supernatant was discarded, a resulting precipitate was air-dried at room temperature for about 30 minutes, and 100 μL of ddH₂O or TE water was added for dissolution to obtain a DNA solution. (2) A corresponding PCR system was prepared, and a corresponding procedure was selected to allow a reaction. Agarose gel electrophoresis was conducted for band analysis. (3) PCR primer sequences were as follows:

LoxP Site PCR Primer Sequences:

MGRIM-19_LoxP_F:
(SEQ ID NO: 1)
5′-CAATTGTCTGATATGGGACCCACGGT-3′ (26 bp)
MGRIM-19_LoxP_R:
(SEQ ID NO: 2)
5′-ATGCTGTACCCTGCAAGAGAAATGAGAC-3′ (28 bp)
A fragment derived from a WT allele has a size of
302 bp and a fragment derived from a mutant allele
has a size of 382 bp.

ATP4b-Cre PCR Primer Sequences:

ATP4b-Cre-F:
(SEQ ID NO: 3)
5′-GCAGATAGCAAGCAAGCTCCAACC-3′
ATP4b-Cre-R:
(SEQ ID NO: 4)
5′-GGATTAACATTCTCCCACCGTCAG-3′
Target band: 800 bp

(4) PCR System

LoxP Site PCR System (20 μL):

| | |
|---|---|
| 2xTag MIX | 10 μL |
| MGRIM-19_LoxP_F | 1 μL (10 μM) |
| MGRIM-19_LoxP_R | 1 μL (10 μM) |
| ddH₂O | 6 μL |
| Template DNA | 2 μL |

ATP4b-Cre PCR System (20 μL)

| | |
|---|---|
| 2xTaq MIX | 10 μL |
| ATP4b-Cre-F | 1 μL(10 μM) |
| ATP4b-Cre-R | 1 μL(10 μM) |
| ddH₂O | 6 μL |
| Template DNA | 2 μL |

(5) PCR Amplification Conditions

| Step# | Temp° C. | Time | Note |
|---|---|---|---|
| 1 | 94 | 2 min | |
| 2 | 94 | 30 sec | |
| 3 | 60 | 30 sec | |
| 4 | 72 | 1 min | |
| 5 | | | 30Times to 2 |
| 6 | 72 | 5 min | |
| 7 | 4 | 99 hrs | |
| 8 | | | End |

(6) A PCR product was identified by agarose gel electrophoresis and the identification results of the genotype of the GRIM-19^{-/-}/ATP4b-cre mouse were shown in FIG. 2B. As shown in FIG. 2A, a cre recombinase was expressed in gastric mucosal parietal cells of the ATP4b-cre mouse, the Loxp site was identified, and exon 3 of the GRIM-19 gene was deleted.

### Example 2 Analysis of Expression of GRIM-19 in a Mouse Gastric Mucosal Tissue

The expression of GRIM-19 in the mouse gastric mucosal tissue was analyzed by WB and immunofluorescence techniques.

WB: Tissue protein from mouse gastric mucosal was extracted using tissue lysis buffers (Beyotime), and then subjected to homogenization on ice for at least 30 minutes, and centrifuged at 12,000 g and 4° C. for 3 minutes, and a supernatant was collected in an Ep tube; protein concentration in the supernatant was measured by a BCA protein concentration determination kit (Beyotime); and a corresponding volume of a sodium dodecyl sulfate-polyacrylamide gel electrophoresis (SDS-PAGE) protein loading buffer (5×) was added to the remaining supernatant, and a resulting mixture was thoroughly mixed, heated at 100° C. for 10 minutes, centrifuged, and then immediately used or stored in a −80° C. freezer. SDS-PAGE: Gel preparation and sample loading: A 12.5% SDS-PAGE separated gel and a 5% SDS-PAGE concentrated gel were adopted. The concentrated gel was run at a constant pressure of 80 V and the separated gel was run at a constant pressure of 120 V until bromophenol blue (BPB) swam to a bottom of the gel. Membrane transfer (wet membrane transfer): With polyvinylidene fluoride (PVDF) as a solid-phase carrier, membrane transfer was conducted at 230 mA for 50 minutes, then a membrane was taken out, and when it was observed that a marker was transferred on the membrane, the membrane was washed with TBST 2 times for 5 minutes each time. Blocking and antibody incubation: The membrane was blocked with 5% BSA (diluted with TBST) and then incubated on a shaker at room temperature for about 1 h. Addition of a primary antibody with a corresponding concentration: (3-actin (sigma, 1:10,000) and GRIM-19 (Santa Cruz, 1:300) were added, and the membrane was incubated on a shaker at 4° C. overnight. The primary antibody was recovered, and the membrane was rinsed with TBST 3 times for 10 minutes, 5 minutes, and 5 minutes, respectively. A corresponding secondary antibody diluted with TBST (1:5,000) was incubated with the membrane at room temperature on a shaker for 1.5 h, then rinsed with TB ST 3 times, and then subjected to exposure.

IFA: The mouse gastric mucosal tissue was embedded with an OCT embedding agent and then stored in a −80° C. refrigerator, a frozen tissue was taken out, then continuously sectioned into 8 μm sections, fixed with 4% PFA for 30 minutes, and washed with PBS three times for 5 minutes each time. A blocking reagent diluted with a mixture of goat serum and PBS in a ratio of 1:1 was added to block at room temperature for 1 h. A primary antibody diluted with an IFA blocking solution (Beyotime) was incubated with the section at 4° C. overnight. The section was washed with PBS three times for 5 minutes each time, an appropriate secondary antibody was selected according to a source of the primary antibody, diluted with an IFA blocking solution according to a ratio of 1:200, and incubated with the section at room temperature in the dark for 1 h, and then washed with PBS 3 times for 5 minutes each time. Counterstaining was conducted with DAPI (final concentration: 1 μg/mL) at room temperature for 10 min, mounting was conducted with an anti-fluorescence quenching mounting solution (purchased from Solarbio), and a resulting section was stored in a wet box at 4° C. in the dark before detection. Images were acquired by confocal laser scanning microscopy (CLSM).

The expression of GRIM-19 in a mouse gastric mucosal tissue was analyzed by the above experimental technique, and results were shown in FIGS. 3A-3C. The control group was GRIM-19^{flox/flox} the heterozygous group was GRIM-19^{flox/flox}/ATP4b-cre, and the homozygous knockout group was GRIM-19^{-/-}/ATP4b-cre. It can be seen from FIGS. 3A-3C that, compared with the control group, the expression of GRIM-19 was reduced in the knockout group, indicating that the mouse modeling was successful.

### Example 3 HE Staining Analysis of a Mouse Gastric Mucosal Tissue

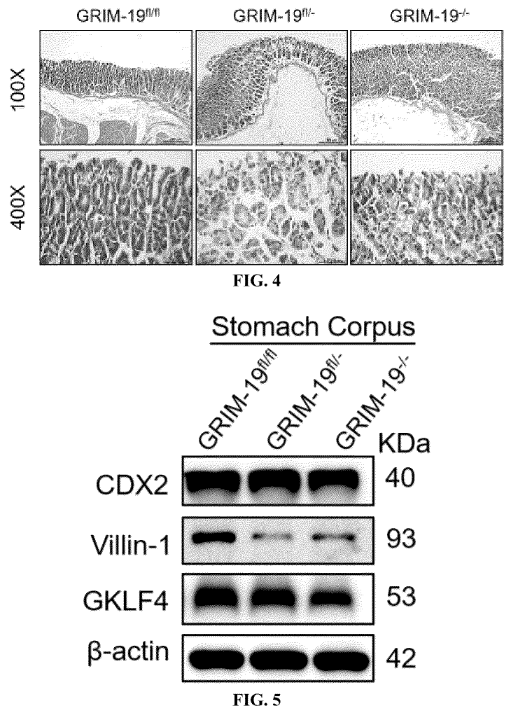

A mouse gastric mucosal tissue was collected from mice in the 8-month-old control group, heterozygous group, and homozygous group. (1) A tissue sample was fixed with a 4% paraformaldehyde solution for at least 24 h. (2) The tissue sample was dehydrated with ethanol at different concentrations, permeabilized with xylene, embedded with paraffin, sectioned into 4 μm sections, and baked at 60° C. for 24 h to 48 h. (3) Dewaxing: The sections were dewaxed with xylene I for 15 minutes and then dewaxed with xylene II for 15 minutes. (4) Hydration: The sections were hydrated with 100% ethanol for 5 minutes, with 95% ethanol for 3 minutes, with 80% ethanol for 3 minutes, and with 75% ethanol for 3 minutes, then rinsed with tap water for 2 minutes, and drained. (5) The sections were soaked in a hematoxylin stain solution for 3 minutes and then rinsed with tap water for 1 minute. (6) The sections were soaked in 1% hydrochloric acid-ethanol for 3 s to 5 s and then rinsed with tap water for 1 minute. (7) The sections were soaked in a saturated lithium carbonate solution for 5 s to 10 s, then rinsed with tap water for 1 min, and then soaked in 95% ethanol for 1 minute. (8) The sections were soaked in an HE stain for 3 s, rinsed with tap water for 1 min, and then soaked in 95% ethanol for 3 s to 10 s, in 100% ethanol I for 1 min, and in 100% ethanol II for 2 minutes. (9) The sections were permeabilized with xylene for 5 minutes and then mounted with a neutral resin. HE staining results were shown in FIG. 4. There were no obvious IM pathological changes of the experimental group compared with the control group, but a mouse gastric mucosa of the knockout group showed an obvious pathological thickening symptom.

### Example 4 Analysis of Expression of IM Markers in a Mouse Gastric Mucosal Tissue

The expression of IM expression markers CDX2, Villin-1, and GKLF4 in the mouse gastric mucosal tissue was detected by WB. As shown in FIG. 5, compared with the control group, there was no significant change of mouse CDX2 in the knockout group, and the expression of Villin-1 and GKLF4 was down-regulated, indicating that the mouse did not undergo an IM lesion at this time point.

### Example 5 Analysis of Expression of SPEM Markers in a Mouse Gastric Mucosal Tissue

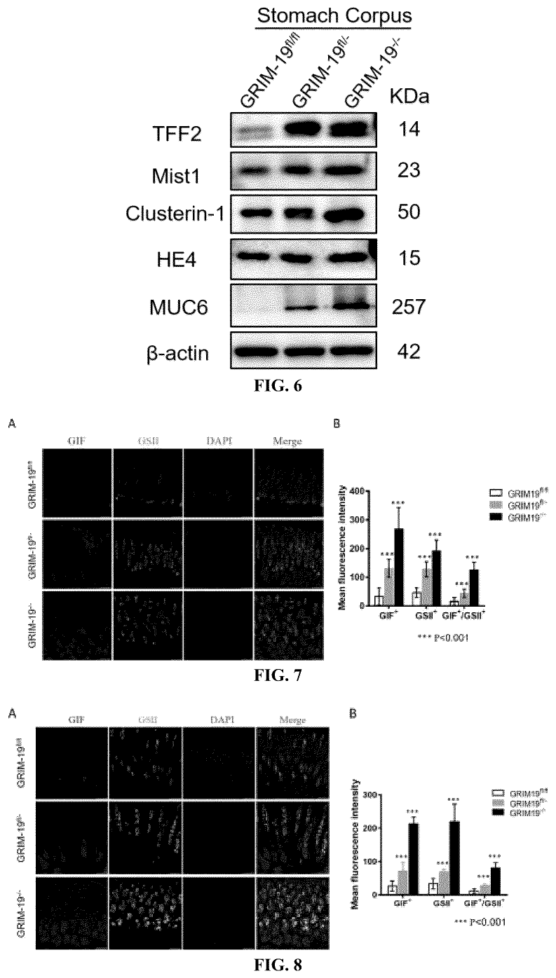

The expression of SPEM markers in the mouse gastric mucosal tissue was detected by WB. Results were shown in FIG. 6. Compared with the control group, the expression of TFF2, Mist1, Clusterin-1, HE4, and MUC6 in the knockout group was enhanced, indicating that, after the specific knockout of GRIM-19, the mouse gastric mucosal tissue showed SPEM pathological changes, which were very significant in the homozygous knockout mouse.

### Example 6 GIF and GSII Colocalization Analysis of SPEM Markers in a Mouse Gastric Mucosal Tissue

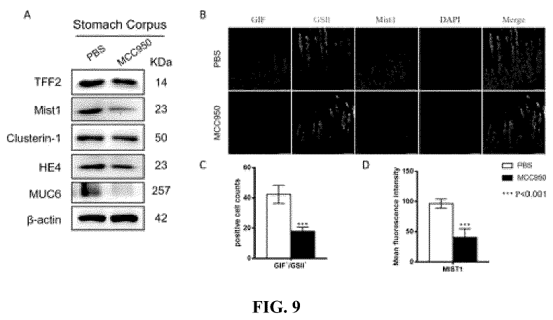

The DIFA technique was used to analyze the GIF and GSII double positive colocalization expression of SPEM markers in the mouse gastric mucosa. Results were shown in FIGS. 7A-7B. Compared with the control group, the number of GIF or GSII single-positive cells in the knockout group was increased significantly, and the number of GIF+/GSII+double-positive SPEM cells was increased significantly, which was especially significant in the homozygous knockout group, indicating that the specific knockout of the GRIM-19 gene could induce the spontaneous pathological formation of SPEM in mice. In order to prove the stability of the modeling method, 4-month-old mice were further taken to conduct GIF and GSII double-standard colocalization analysis of SPEM markers, and results were shown in FIGS. 8A-8B. The results showed that the pathological phenomenon of SPEM could also occur in the 4-month-old knockout mice, indicating that the construction of this model had excellent stability.

### Example 7 Analysis of SPEM in GRIM-19 Homozygous Knockout Mice Intervened with MCC950

It is known that the inflammasome inhibitor MCC950 can significantly inhibit the expression of the NLRP3 inflammasome. In order to demonstrate the simulation of the SPEM model and a therapeutic effect of a drug for the model, the GRIM-19^{-/-}/ATP4b-cre homozygous knockout mouse was subjected to in vivo intervention with MCC950, and the control group was injected with an equal amount of PBS. MCC950 was purchased from MCE and was administered in vivo at a concentration of 10 mg/kg. MCC950 was injected three times a week continuously for four weeks to obtain a gastric mucosal tissue.

The expression of SPEM markers in the mouse gastric mucosal tissue was detected by WB. Results were shown in FIG. 9A. Compared with the PBS control group, the expression of TFF2, Mist1, Clusterin-1, HE4, and MUC6 was inhibited in the MCC950 drug treatment group, indicating that the inhibition of inflammation in mice could slow down the pathological occurrence of SPEM.

The IFA technique was further used to analyze the GIF, GSII, and Mist1 localization expression of SPEM markers in the mouse gastric mucosa. Results were shown in FIG. 9B. Compared with the PBS control group, the GIF and GSII double-positive cells were significantly reduced and the expression of Mist1 was also significantly reduced in the drug treatment group, indicating that, after the in vivo intervention by MCC950, SPEM cells in the homozygous knockout mouse were significantly reduced and the pathological phenotype of SPEM was effectively alleviated; and the constructed model could well simulate the occurrence and development of SPEM.

In summary, the mouse gastric mucosal parietal cell-specific knockout of the GRIM-19 gene in the present disclosure can induce spontaneous SPEM.

What is claimed is:

1. (canceled)

2. (canceled)

3. (canceled)

4. (canceled)

5. (canceled)

6. (canceled)

7. A simulation method for a chronic atrophic gastritis (CAG) lesion, comprising:
taking a metaplasia lesion stage as a simulation object; selecting a simulation form of a spasmolytic polypeptide-expressing metaplasia (SPEM); and conditionally deleting a gene associated with retinoid-IFN-induced mortality-19 (GRIM-19) from gastric mucosal parietal cells;
the simulation method comprises the following steps:
(1) determining a target gene by determining 5 exons of a mouse chromosome 8, where mouse GRIM-19 is located on the mouse chromosome 8, and selecting exon 3 as a conditional knockout region;
(2) designing and constructing a targeting vector plasmid by providing a bacterial artificial chromosome (BAC) clone RP23-74A9 or RP23-114L20 from a C57BL/6J library as a template, preparing a homology arm and a conditional knockout (CKO) region through polymerase chain reaction (PCR); and
in a targeting vector, providing a flank of a neomycin (NEO) cassette as a Frt site and a flank of the CKO region as a LoxP site, using diphtheria toxin A (DTA) for a negative selection;
(3) subjecting an embryonic stem (ES) cell to an electroporation and a positive clone screening, microinjecting a resulting ES cell into a mouse to prepare a chimera mouse, intercrossing the chimera mouse and a flp mouse, and deleting a NEO resistance gene out to obtain a GRIM-19^{flox/-}F1 mouse; and
subjecting the GRIM-19^{flox/-}F1 mouse to a first propagation to obtain an offspring mouse, identifying, and raising the offspring mouse together with a wild-type (WT) mouse C57B/L6 in a first cage to allow a second propagation to obtain a GRIM-19^{flox/flox} homozygous mouse; and
(4) intercrossing the GRIM-19^{flox/flox} homozygous mouse with an ATP4b-cre mouse in a second cage, identifying to obtain a GRIM-19^{flox/flox}/ATP4b-cre mouse, and establishing a gastric mucosa-specific parietal cell GRIM-19-knockout mouse strain GRIM-19^{-/-}/ATP4b-cre.
